(19) United States
(12) Patent Application Publication
(10) Pub. No.: US 2026/0163413 A1
(43) Pub. Date: Jun. 11, 2026

(54) USER-GUIDED SYSTEMS AND METHODS FOR ALIGNING A CHARGER WITH A RECHARGEABLE DEVICE

(71) Applicant: THE ALFRED E. MANN FOUNDATION FOR SCIENTIFIC RESEARCH, VALENCIA, CA (US)

(72) Inventors: Rodney Faris, Fillmore, CA (US); Susanna M. Lin, Los Angeles, CA (US)

(73) Assignee: THE ALFRED E. MANN FOUNDATION FOR SCIENTIFIC RESEARCH, VALENCIA, CA (US)

(21) Appl. No.: 19/412,208

(22) Filed: Dec. 8, 2025

Related U.S. Application Data
(60) Provisional application No. 63/729,626, filed on Dec. 9, 2024.

(51) Int. Cl. H02J 50/90; A61N 1/378; H02J 50/10; H02J 50/40

(52) U.S. Cl. CPC H02J 50/90; A61N 1/3787; H02J 50/10; H02J 50/402

(57) ABSTRACT

A wireless charging system and associated methods are provided to assist a user with manually aligning a wireless charger with an implantable device, such as an implantable medical device (“IMD”). Both the charger and the IMD are equipped with at least one inertial sensor (e.g., an accelerometer, a gyroscope, or a magnetometer). Data from these sensors is wirelessly sent to a controller, which processes the information to generate three-dimensional (3-D) digital models (also known as “digital twins”) of the charger and IMD on a graphical user interface, showing their relative position and orientation in real-time. During alignment, a patient initially positions the charger near the IMD. The digital twins are displayed to the patient as visual aids, helping the patient manually adjust the charger into parallel and axial alignment with the IMD quickly and precisely, thereby enhancing user experience and wireless charging efficiency.

### CROSS-REFERENCE TO RELATED APPLICATIONS

The present disclosure relates to and claims priority from U.S. provisional patent application number 63/729,626, filed on Dec. 9, 2024, and entitled “User-Guided Systems and Methods for Aligning a Charger with a Rechargeable Device”.

### FIELD

The present disclosure generally relates to a wireless charging system, and more particularly to a method and apparatus for aligning a wireless power transmitting coil with the receiving coil of a visually obscured rechargeable device, such as an implantable medical device (IMD).

### BACKGROUND

Electromagnetic induction has been widely adopted for wireless charging across various industries and devices, including electric vehicles, consumer electronics, and implantable medical devices (IMDs). In conventional inductive charging systems, an alternating current (AC) is supplied to a transmitting coil, which generates an oscillating magnetic field. This magnetic field can induce a corresponding current in the receiving coil of a nearby rechargeable device, enabling wireless energy and data transfer without physical connectors.

Despite its advantages, such as eliminating wired interfaces and improving device sealing, inductive charging systems have certain limitations. A primary challenge lies in the sensitive relationship between power transfer efficiency and coil alignment. Misalignment between the transmitting and receiving coils significantly reduces the amount of current induced in the receiving coil, resulting in slower charging and energy losses.

These limitations are particularly pronounced in IMD applications. Once implanted, an IMD may undergo slight positional changes within the body due to movement, variations in posture, or fluctuations in the patient's weight. Such changes can alter the orientation or tilt of the IMD relative to the skin surface, making it difficult to achieve precise and repeatable alignment with an external charger. Because the IMD is not externally visible, patients cannot readily confirm proper alignment, which can lead to inefficient charging, shortened battery life, and excessive heat buildup.

### SUMMARY

This summary section introduces non-limiting, non-exhaustive examples of the present disclosure and is not intended to limit the scope of the claims.

Systems and methods for aligning a wireless charger with a rechargeable device stand to be improved. Conventional wireless charging systems fail to provide a simple, reliable method for achieving parallel and axial alignment between a wireless charger and a visually obscured device, such as an implantable medical device (IMD). This can deleteriously affect device lifespan and system usability. In exemplary embodiments, the rechargeable device comprises an implantable medical device (IMD). However, aspects of the present disclosure are not limited solely to medical devices and could apply to other applications.

In one aspect, this disclosure provides a wireless power transfer system for enabling precise alignment between a wireless charger and an IMD. The system includes a charger having a power transmitting coil (Tx coil), and an IMD with a power receiving coil (Rx coil). The Tx coil is contained within a housing and electrically connected to a power source. When energized, the Tx coil generates a magnetic field for inductive coupling with the Rx coil, enabling wireless power transfer and optional telemetry between devices.

In another aspect, the system is configured to enable accurate manual alignment of a charger with an IMD. The system includes first and second sensors, one associated with the charger and the other with the IMD. The sensors are used, in part, to collect inertial data indicative of each device's position and orientation (or “pose”) in three-dimensional space. By continuously processing sensor data, movements of the charger and IMD can be tracked in real time. To that end, the system includes one or more processors for processing the sensor data and tracking device movements. This disclosure defines a “processor” as one or more hardware, software, middleware, or firmware elements capable of sending instructions and receiving data from other system components. The processor can execute instructions stored in memory, which may be associated with the processor or another system component.

Any sensor within the system (i.e., either of the first or second sensors) could comprise one or a plurality of sensors. In exemplary embodiments, the first sensor (of the charger) and the second sensor (of the IMD) each include an inertial measurement unit (IMU). The IMU includes one or more inertial sensors selected from: an accelerometer, a gyroscope, and/or a magnetometer. Data collected from these and other sensors may be fused (computationally combined using, e.g., an Extended Kalman Filter, executed by a processor) to improve the precision and reliability of motion tracking. Inertial data from such sensors can also be readily transformed into quaternions to facilitate lightweight processing, reference frame alignment, and the generation of an alignment indicator on a display.

Any device included within the system can include a communication unit for wirelessly sending or receiving data to or from another system device. The communication unit could comprise any known type of radio frequency (RF) transceiver capable of wirelessly sending or receiving data in accordance with transmission and encryption protocols suitable for medical devices. Examples include Bluetooth, Bluetooth LE, Wi-Fi, Ultra-Wideband (UWB), and MedRadio. Any communication unit within the system could consist of one or more antennas.

In another aspect, the system is configured to process sensor data to generate a digital alignment indicator on a display or graphical user interface (GUI). The alignment indicator provides visual feedback to the user and guides manual repositioning of the charger into parallel and/or axial alignment with the IMD.

In exemplary embodiments, the alignment indicator includes “digital twins” of the IMD and charger, generated on a display. The digital twins comprise real-time, three-dimensional digital representations of the charger and the IMD. They mirror the orientation, position, and movements of their physical counterparts and update in real time as the physical devices are moved. By referencing the display while manipulating the charger, a user can achieve rapid and precise charging alignment. This enhances user experience and power transfer efficiency.

In some examples, the alignment indicator includes written alignment instructions, generated by the processor, and displayed to guide manual repositioning of the charger into parallel and axial alignment with the IMD. Such instructions may include messages such as “Move the charger 3 mm left”, “Slowly rotate the charger clockwise”, or “Tilt the top edge of the charger towards your body”, etc.

In some examples, the system includes an external controller with a processor and a display. During an alignment process, sensor data is transmitted from the IMD and charger to the controller. The controller receives and processes the data to generate digital twins of the charger and the IMD on a GUI, showing their positions and orientations in real time, thereby providing a visual reference for a user to precisely align the devices.

In various examples, the controller could comprise a dedicated medical device, such as a patient remote or clinician programmer, or a commercial electronic device, such as a smartphone or tablet. In some implementations, the controller could be integrated with or coupled to the charger.

In some examples, the charger can be outfitted with components, such as speakers, lights, or mechanical transducers. These components can be used to transmit haptic feedback (vibrations), audio feedback (beeps/chimes), or visual feedback to the user. For instance, a pattern of vibrations, beeps, or lights may be used to guide placement of the charger, indicate proximity to the IMD, or alert the user to a charging status change.

In some examples, at least one sensor in the system is configured to monitor charge transfer efficiency (CTE). Functionally, the system can process CTE measurements alongside inertial data to more precisely estimate (e.g., triangulate) the IMD's position. For instance, measurements of CTE may be collected, timestamped, and fused with inertial data from the charger. As a user scans the charger over a bodily area containing the implant, CTE and motion data are collected continuously. A processor analyzes the data to identify a location corresponding to a relative maximum CTE measurement, which corresponds to the IMD's location. Next, the processor analyzes the charger's recent motion data to compute a distance or a direction to the IMD. This is used to generate an alignment indicator on a display.

Depending on the example, one or more CTE sensors could be provided in the charger, the IMD, or both. However, because minimizing the IMD's power consumption is often preferable, CTE sensing could be performed exclusively by one or more sensors in the charger, connected to the Tx coil. In one such example, a CTE sensor in the charger is configured to detect either the magnitude or phase of current in the Tx coil. Such measurements can be used to infer the strength or quality of an inductive coupling. In other instances, a CTE sensor in the IMD could be used to detect either the rectified output voltage or the current flow between the Rx coil rectifier and a battery. Such measurements can be used to quantify the amount or rate of power received.

In some examples, when an RF signal is transmitted from the IMD to the charger, the charger is configured to record one or more RF signal metrics, such as received signal strength (RSSI), phase angle, or time of arrival (ToA). Conventional RF ranging methods use one or more of these metrics to obtain a rough estimate of the distance or direction between devices but lack the granularity to enable precise alignment between an IMD and a charger. However, systems of this disclosure are configured to process RF signal metrics in conjunction with IMU or CTE data, optionally fusing the data from each, thereby enhancing the system's ability to track motion smoothly and determine position. Further, periodic ranging from an RF signal may be used to calibrate the inertial sensors or associate inertial reference frames of the IMD and charger.

In some examples, detection of a known device identifier and an RSSI exceeding a predetermined threshold triggers activation of the first and second sensors. By employing this approach, power is supplied to the system sensors only when the IMD and a trusted charger are in proximity, rather than when they are a substantial distance apart. This targeted activation helps ensure that energy is not wasted by either device powering their sensors when sensor-based alignment is not required. Such methods also enhance data security by restricting the transfer of sensitive data to instances when a trusted device is in valid proximity.

Beyond electronic and software solutions for alignment, mechanical aids can further improve user experience. For example, the system may include a mount. The mount is configured for assembly with the charger. It accommodates manual adjustments to position and orient the charger in a desired pose for alignment. In exemplary embodiments, the mount allows the charger to be tilted, suspended at an angle above the skin surface, enabling parallel alignment between the charger and the IMD even if the IMD is offset from the skin surface.

The mount usually features a movable coupling, like a ball joint or hinge, between an upper platform (attached to the charger) and a base (that interfaces with the user's body or clothing). The mount may attach to the charger with snap-fit retention features or be built directly into the charger housing. The base can be temporarily secured to the user with adhesives or hook-and-loop fasteners, providing stability and comfort during charging.

In some examples, the platform is spaced apart from the base by a shank. An upper edge of the shank may be fixed to a lower surface of the platform or formed integrally therewith. The shank extends between the platform and the base, coupling to the base at a ball joint or hinge disposed on the base's upper surface. The base includes a lower surface disposed opposite the upper surface. The lower surface is adapted to interface with the user's body or clothing and contains means for releasable attachment to the user. The attachment means can comprise an adhesive, hook-and-loop fasteners, or any other known method for temporarily affixing objects to the human body.

In some examples, the IMD comprises an implantable pulse generator (IPG) equipped with stimulation circuitry. This stimulation circuitry is securely housed within a case and electrically communicates with the receiving coil or an intermediary battery. The stimulation circuitry incorporates one or more hardware and/or software elements designed to execute specific algorithms that govern the delivery of stimulation (e.g., one or more pulses of electrical current) to a patient's biological tissue. Such stimulation may be directed to one or more nerves, muscles, or other tissues as required for therapeutic purposes. The IPG may further include a stimulation lead, which extends outward from the case and serves as an electrical conduit between the stimulation circuitry and at least one electrode. This arrangement enables the precise delivery of therapeutic stimulation through the electrode to a desired location in the body.

Any aspect, feature, advantage, or component discussed above could be interchangeably applied between the examples, and no single aspect, feature, or component should be interpreted as being essential unless otherwise stated. Various other features and advantages will be described in later portions of this disclosure.

### BRIEF DESCRIPTION OF THE DRAWINGS

Detailed descriptions of exemplary embodiments will be made with reference to the accompanying drawings. In some instances, well-known structures and components are shown in block diagram form in order to avoid obscuring such concepts.

FIGS. 1a-1b illustrate a wireless power transfer system that includes a rechargeable implanted medical device (IMD) and a charger, in accordance with some examples.

FIG. 2 is a schematic of a wireless power transfer system, in accordance with some examples.

FIG. 3 is a front view of an IMD implanted within the chest region of a subject.

FIG. 4 is a front view of the subject of FIG. 3 donning a charger.

FIG. 5 is a rear view of an IMD implanted in the lower-back region of a subject.

FIG. 6 is a rear view of the subject of FIG. 5 donning a charger.

FIG. 7 is a front view of the subject of FIG. 6.

FIG. 8 is a front view of a transmitting coil in alignment with a receiving coil, according to some examples.

FIG. 9 is a front view of a transmitting coil that is misaligned with a receiving coil, according to some examples.

FIG. 10 is a front view of a transmitting coil that is misaligned with a receiving coil, according to some examples.

FIGS. 11a-11d (collectively FIG. 11) is a first example of a mount for manually adjusting the disposition of a charger.

FIGS. 12a-12b (collectively FIG. 12) is a second example of a mount for manually adjusting the disposition of a charger.

FIG. 13 is a third example of a mount for manually adjusting the disposition of a charger.

FIG. 14 is a subject conducting a step for calibrating inertial sensors, according to some examples.

FIG. 15 is a subject conducting another step for calibrating inertial sensors, according to some examples.

FIG. 16 is a subject conducting yet another step for calibrating inertial sensors, according to some examples.

FIG. 17 is a front view of a charger being scanned across the subject's chest around the IMD.

FIG. 18 is a flow diagram outlining the general functionality of a sensor fusion processor, according to some examples.

FIG. 19 is a digital display on a controller depicting the disposition of a charger relative to a rechargeable device.

FIG. 20 is the display of FIG. 19 after the charger has been tilted into parallel alignment with the rechargeable device.

FIG. 21 is the display of FIG. 20 after the charger has been tilted into positioned centrally over the rechargeable device.

FIG. 22 is the display of FIG. 21 after the charger has been rotated into alignment with the rechargeable device.

FIG. 23 is a flowchart depicting steps in accordance with a method for aligning a wireless power transfer system, according to some examples.

FIG. 24 is a flowchart depicting steps of a computer-implemented method for collecting inertial data to generate digital twins, according to some examples.

FIG. 25 is a flowchart depicting steps of a computer-implemented method for collecting charge transfer efficiency (CTE) data, according to some examples.

FIG. 26 is a flowchart depicting steps of a computer-implemented method for collecting radio frequency (RF) data metrics to aid in the determination of a distance or direction between a charger and an IMD, according to some examples.

### DETAILED DESCRIPTION

The following is a detailed description of the best-known modes of carrying out the invention. This description is not to be taken in a limiting sense but is made merely for the purpose of illustrating the general principles. In the drawings, the relative sizes of elements, layers, and regions may be exaggerated for clarity. In some cases, well-known structures and components are shown in block diagrams to avoid obscuring these concepts. Unless otherwise noted, like reference numerals denote like elements and, thus, descriptions thereof may not be repeated.

The present disclosure provides systems and methods for aligning a wireless power transfer device, such as a wireless charger, with a visually obscured rechargeable device, such as an implantable medical device (IMD), to improve the efficiency of inductive charging and/or data transfer. The systems and methods are user-guided, presenting a user with three-dimensional digital images (i.e., “digital twins”) of the charger and IMD on a graphical user interface (GUI) or display. The position and orientation of the digital twins adjust dynamically (e.g., in real time) as the user moves the charger, providing a visual aid to help the user determine when the devices are correctly aligned. This is accomplished, at least in part, by processing data collected by inertial sensors within the charger and the IMD. The system may also provide a user with guided alignment instructions, including visual cues, sounds, prompts, and/or haptic feedback to guide the user toward precise tri-planar alignment (i.e., alignment along each of the x, y, and z planes). Written alignment instructions may also be displayed to the user. In some examples, a mount is provided for fixing the charger in a desired position and orientation for charging.

### Definitions

The terminology used herein is for the purpose of describing particular example embodiments only and is not intended to be limiting with respect to the present disclosure.

As used herein, the terms “pose” and “disposition” may be used interchangeably to refer to an object's location, position, and orientation in three-dimensional (3D) space, and, in some instances, the “disposition” of an object may refer to its position and/or orientation relative to another object or reference point. Accordingly, the term “disposition” or “pose”, when made with reference to a singular object, may encompass the object's location (x, y, z), angular orientation (roll, pitch, yaw), and heading in 3D space. Such determinations could be made with respect to a global reference frame (e.g., Earth's reference frame) or a local reference frame. Further, the “disposition” or “pose” of one object relative to another can indicate their relative alignment in 3D space.

As used herein, the singular forms “a” and “an” are intended to include the plural forms as well, unless the context clearly indicates otherwise. It will be further understood that the terms “comprises,” “comprising,” “includes,” and “including,” when used in this specification, specify the presence of the stated features, integers, acts, operations, elements, and/or components, but do not preclude the presence or addition of one or more other features, integers, acts, operations, elements, components, and/or groups thereof. As used herein, the term “and/or” includes any combination of one or more of the associated listed items. Expressions such as “at least one of,” when preceding a list of elements, modify the entire list of elements and do not modify the individual elements of the list.

Although the terms “first,” “second,” “third,” etc., may be used herein to describe various elements, components, regions, layers, and/or sections, these elements, components, regions, layers, and/or sections should not be limited by these terms. These terms are used to distinguish one element, component, region, layer, or section from another element, component, region, layer, or section. Thus, a first element, component, region, layer, or section described below could be termed a second element, component, region, layer, or section, without departing from the spirit and scope of the present disclosure.

Spatially relative terms, such as “beneath,” “below,” “lower,” “under,” “above,” “upper,” and the like, may be used herein for ease of explanation to describe one element or feature's relationship to another element(s) or feature(s) as illustrated in the figures. It will be understood that the spatially relative terms are intended to encompass different orientations of the device in use or in operation, in addition to the orientation depicted in the figures. For example, if the device in the figures is turned over, elements described as “below” or “beneath” or “under” other elements or features would then be oriented “above” the other elements or features. Thus, the example terms “below” and “under” can encompass both an orientation of above and below. The device may be otherwise oriented (e.g., rotated 90 degrees or at other orientations) and the spatially relative descriptors used herein should be interpreted accordingly.

It will be understood that when an element or layer is referred to as being “on”, “connected to”, “coupled to”, or “adjacent to” another element or layer, it can be directly on, connected to, coupled to, or adjacent to the other element or layer, or one or more intervening element(s) or layer(s) may be present. In contrast, when an element or layer is referred to as being “directly on,” “directly connected to”, “directly coupled to”, or “immediately adjacent to” another element or layer, there are no intervening elements or layers present.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The terms “about” or “substantially”, may be used herein to indicate a degree of uncertainty in measurement equivalent ±10% or, when made with reference to an angle, ±10 degrees. For instance, the term “substantially parallel” may refer to two or more devices having at least one alignment plane that is angularly offset by ≤10 degrees.

The electronic or electric devices and/or any other relevant devices or components, as described in the present disclosure, may be implemented using any suitable hardware, firmware (e.g., an application-specific integrated circuit), software, or a combination of software, firmware, and hardware. For example, the various components of these devices may be formed on a single integrated circuit (IC) chip, on separate IC chips, on a flexible printed circuit film, on a tape carrier package (TCP), on a printed circuit board (PCB), or on a single substrate. Further, the various components of these devices may be a process or thread, running on one or more processors, in one or more computing devices, executing computer program instructions and interacting with other system components for performing the various functionalities described herein. The computer program instructions may be stored in a memory that can be implemented in a computing device using a memory device, such as random-access memory (RAM). The computer program instructions may also be stored on other non-transitory computer-readable media, such as a CD-ROM, flash drive, or the like. Also, a person of skill in the art should recognize that the functionality of various computing devices may be combined or integrated into a single computing device, or the functionality of a particular computing device may be distributed across one or more other computing devices without departing from the spirit and scope of the example embodiments of the present disclosure.

By way of example, an element, or any portion of an element, or any combination of elements may be implemented as a “processing system” or “controller” that includes one or more processors.

As used herein, the term “processor” includes any combination of hardware, firmware, memory and software, employed to process data or digital signals. The hardware of a controller may include, for example, a microcontroller, a microprocessor, application specific integrated circuits (ASICs), general purpose or special purpose central processors (CPUs), digital signal processors (DSPs), graphics processors (GPUs), and programmable logic devices such as field programmable gate arrays (FPGAs). In a processor, as utilized herein, each function is performed either by hardware configured, i.e., hard-wired, to perform that function, or by more general purpose hardware, such as a CPU, configured to execute instructions stored in a non-transitory storage medium or memory. A processor may contain two or more processors, for example, a processor may include two processors, an FPGA and a CPU, interconnected on a PCB.

One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, algorithms, functions, etc., whether referred to as software, firmware, middleware, microcode, hardware description language, or otherwise.

In this document, one or more processors may be described as being capable of performing “sensor fusion”. Sensor fusion is the process of combining data from multiple sensors to produce information that is more accurate, reliable, and comprehensive than that from any single sensor. It typically involves algorithms that integrate heterogeneous sensor inputs to reduce uncertainty, improve robustness, and enhance situational awareness. Such algorithms can include, for example, a Kalman filter, an extended Kalman filter, or an unscented Kalman filter, among others.

As used herein, descriptions of radio frequency (RF) signal metrics, such as, for example, Received Signal Strength Indicator (RSSI) and Angle-of-Arrival (AoA), are defined in accordance with their standard technical definitions. Such metrics may be applicable, for example, to a Bluetooth, Wi-Fi, or ultra-wideband (UWB) signal.

### Example Embodiments

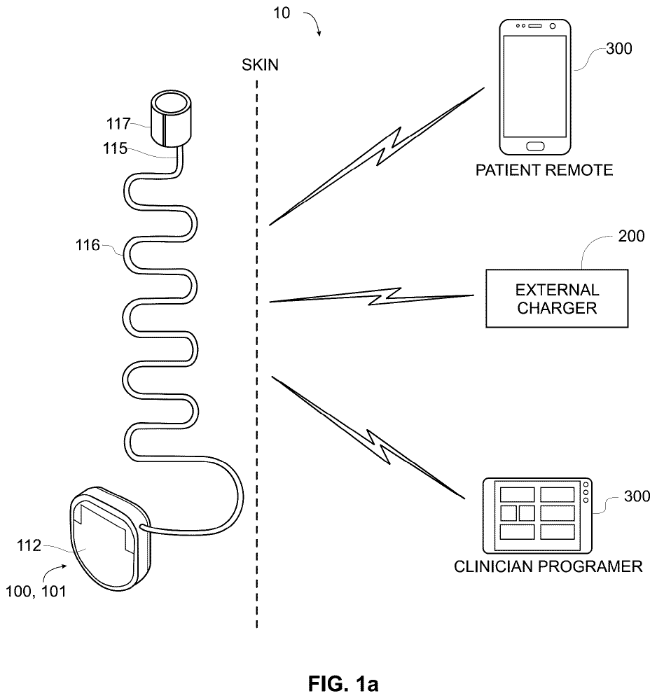

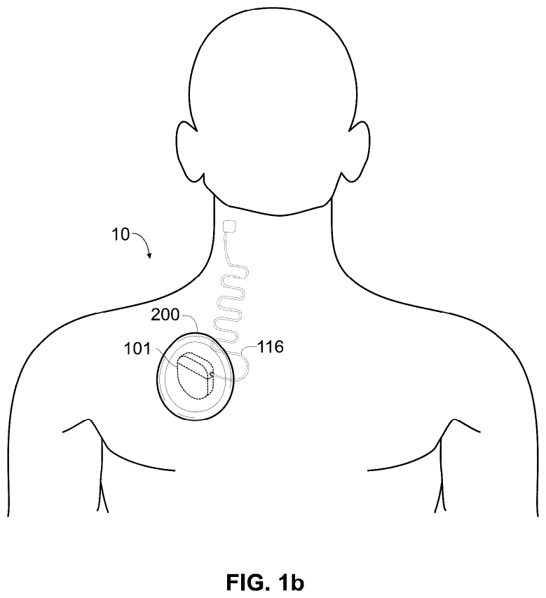

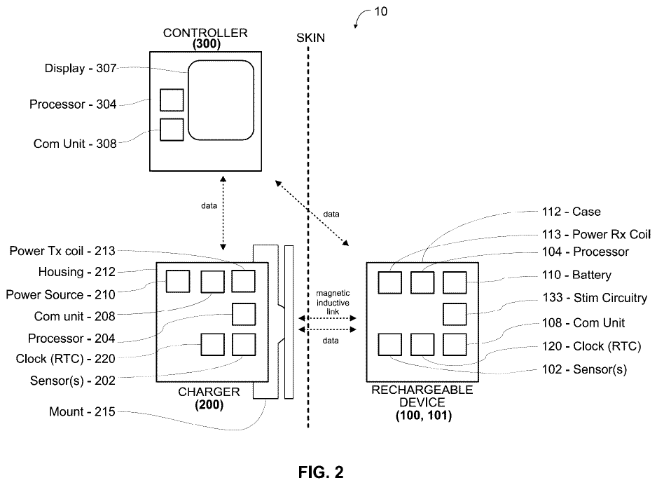

FIGS. 1-2 provide a simplified representation of a wireless power transfer system (“system”) 10 and a schematic illustrating the main components of system 10 in block form.

In the illustrated examples, system 10 comprises three primary elements: a charger 200, a rechargeable device 100, and an external controller (“controller”) 300. In this example, the rechargeable device comprises an IMD 101.

The charger 200 includes a transmitting coil 213. When the charger 200 is activated, a current passes through the transmitting coil 213, generating a magnetic field for inductively coupling with the receiving coil 113 contained within IMD 101. Inductive coupling enables wireless charging of the implanted device and may also be used for data transmission, for example via charge-field modulation.

Controller 300 is configured to communicate wirelessly, such as via Bluetooth, UWB, or Wi-Fi, with at least one of IMD 101 and charger 200. Wireless data or commands can be transferred between them.

In some embodiments, controller 300 could be designed specifically for medical use and may comprise a clinician programmer (CP) 300a or patient remote (PR) 300b. Alternatively, the external controller 300 could be a consumer electronic device, such as a smartphone, tablet, laptop, or wearable device (e.g., a smartwatch). In some cases, controller 300 could be optionally coupled to or integrated within the body of charger 200.

With specific reference to FIGS. 1a-1b (collectively FIG. 1), IMD 101 comprises an implantable pulse generator (IPG) capable of delivering electrical stimulation pulses to the biological tissue, such as a nerve or muscle, of a subject. IMD 101 includes a case 112 containing stimulation circuitry in electrical communication with the proximal end of an implanted stimulation lead 116. The stimulation circuitry incorporates one or more hardware and/or software elements designed to execute specific algorithms that govern the delivery of stimulation (e.g., electrical current pulses) to a patient's biological tissue. Such stimulation may be directed to one or more nerves, muscles, or other tissues as required for therapeutic purposes. Stimulation lead 116 extends outwardly from the case 112 and is electrically coupled to at least one electrode (“electrode”) 117 disposed at or near its distal end 115. Electrode 117 is configured to deliver electrical stimulation pulses from the IPG to neural tissue and, in some examples, to sense physiological data (e.g., neural action potentials).

Efficient power transmission between charger 200 and IMD 101 often requires precise alignment between the charger's transmitting coil 213 and the IMD's receiving coil 113 along three alignment planes (x, y, z). An angular offset as small as 15° between the transmitting and receiving coils (113, 213) can result in no charging, slow charging, excessive heat production, or device malfunction in certain instances. Following IMD implantation, it is typical for the receiving coil 113 to be angularly offset relative to an external charging interface (e.g., the patient's skin or a garment where charger 200 is mounted). The disposition of an implanted IMD is also subject to change over time with postural changes and weight fluctuations of the patient.

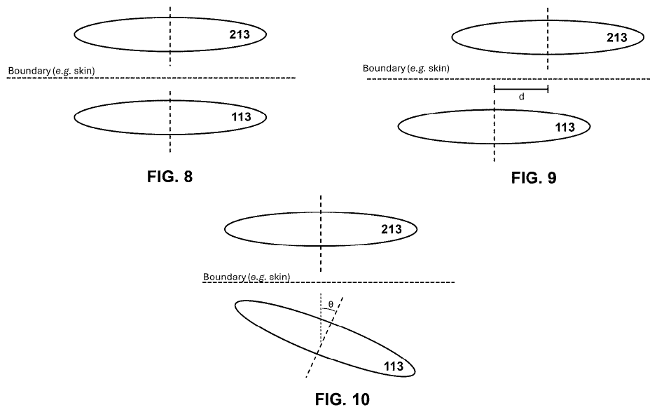

Briefly referring to FIGS. 8-10, simplified views of the transmitting coil 213 and the receiving coil 113 (of IMD 101) are shown in different alignment states. An example of an ideal alignment for charging is shown in FIG. 8, where the transmitting coil 213 and the receiving coil 113 are axially aligned and substantially parallel to each other. In FIGS. 9-10, the transmitting coil 213 and receiving coil 113 are either angularly offset or are spaced apart by some distance “d” and thus may not be optimally aligned for efficient charging. In alternate embodiments incorporating differently shaped coils, suitable alignment for charging might occur when the transmitting coil 213 and receiving coil 113 are positioned differently from the configuration shown (e.g., when the transmitting and receiving coils 213 and 113 are oriented perpendicular to one another). Regardless, those skilled in the art will understand that the general alignment principles and methods described later in this disclosure could still be applied without substantially departing from the underlying concepts.

With returned reference to FIG. 2, the IMD 101 further includes at least one sensor 102, a processor (e.g., a processing circuit) 104, a non-volatile memory, a wireless communication unit 108 (e.g., a radio frequency receiver, transmitter, or transceiver), and a timer 120 (e.g., a real-time clock (RTC)) disposed within the case 112. In this instance, a refillable power supply (e.g., a rechargeable battery) 110 is provided for storing power that is wirelessly received via receiving coil 113. Power supply 110 is not required in all implementations. For instance, in alternate embodiments, power supply 110 may not be included, receiving coil 113 could be directly coupled to stimulation circuitry 133, and the IMD may be configured to deliver stimulation pulses only when actively receiving power from external charger 200.

Sensor 102 is configured, at least in part, to record inertial data that is subsequently processed to determine and/or track the disposition of IMD 101 in 3D space. Sensor 102 may, depending on the example, comprise one or a plurality of sensors, and, for instance, could include any one or a combination of: an accelerometer, a gyroscope, a magnetometer, or an inertial measurement unit (IMU) that consists of any combination of an accelerometer, a gyroscope, or a magnetometer. Sensor 102 can also be used for additional purposes, depending on the specific needs of a particular application. As one such example, sensor 102 could further be utilized for measuring one or more physiological signals of the patient (e.g., respiration rate, heart rate, activity level, body position, etc.). As another example, sensor 102 could be used to monitor “charge transfer efficiency” (CTE) by, for example, monitoring the current or voltage in receiving coil 113.

Communication unit 108 provides wireless communication links through the skin of the patient so that data from IMD 101 may be transmitted to the charger 200 or controller 300. Communication unit 108 comprises one or more RF antennas configured to operate as receivers, transmitters, or transceivers for radio frequency signals. Such wireless links may include Bluetooth™, Bluetooth Low Energy, ultra-wideband, or any other known wireless transmission protocol having suitable authentication and encryption to protect patient data.

In various examples, communication unit 108 is configured to wirelessly transmit inertial data (collected by sensor 102) to either or both charger 200 and controller 300. Following receipt of the inertial data, either charger 200 or controller 300 may be responsible for processing the inertial data to determine (i.e., measure or calculate) the disposition of IMD 101 within the subject. In some instances, communication unit 108 may be configured to periodically transmit an advertising signal containing identifying information, such as a manufacturer part number or a unique device identifier of IMD 101.

Any RF signal transmitted by a communication unit of system 10 could include intrinsic characteristics or “metrics”, such as a received signal strength indicator (RSSI), a phase angle, a time of arrival (ToA), or an angle of arrival (AoA), that a receiving device may optionally store in memory for further processing.

Charger 200 includes a power source 210, e.g., a rechargeable or non-rechargeable battery, or may be configured to receive power from an external source, such as an electrical outlet. Transmitting coil 213 is electrically connected to power source 210, and, when energized, is configured to inductively couple with receiving coil 113 for wireless charging.

Transmitting coil 213 could comprise one or a plurality of transmitting coils 213, depending on the particular embodiment. By way of example and not limitation, additional examples for transmitting coils and details about their construction are shown and described in U.S. patent Application Ser. No. 17/517518, Ser. No. 18/153991, Ser. No. 18/179,821, and/or Ser. No. 18/628,591, each of which is assigned to the present Applicant and incorporated herein by reference in its entirety.

Charger 200 includes at least one sensor 202 within or on housing 212. A processor 204 and a communication unit 208 are also provided, and, in specific examples, an internal memory device may be provided within housing 212. The memory can store data collected by sensor 202, charge delivery parameters, historical pairing data, and charging data, among other information. In some instances, additional components, such as a speaker, a light, or piezoelectric transducers, may be included within or on the charger housing 212 to provide audio, visual, or haptic feedback to the user during the alignment process.

Sensor 202 (of charger 200) may be similar to sensor 102 (as previously described in relation to IMD 101). For instance, sensor 202 may comprise one or more sensors and includes at least one inertial sensor selected from: an accelerometer, a gyroscope, a magnetometer, or an IMU. Sensor 202 is configured, at least in part, for collecting inertial data that is subsequently processed to determine (i.e., calculate or measure) the charger's position and orientation in 3D space (henceforth referred to as the “pose” or “disposition” of charger 200). In many cases, it can be advantageous for charger sensor 202 to include one or more inertial sensors of the same variety/type as IMD sensor 102. This can simplify subsequent calculations and/or comparisons of their respective inertial data to determine their relative alignment in 3D space. Sensor 202 could also be configured to monitor charge transfer efficiency (CTE) by detecting either current or voltage in the transmitting coil 213.

Communication unit 208 enables charger 200 to wirelessly send and/or receive data with other devices in system 10. Communication unit 208 may be technically similar to communication unit 108 (previously described in relation to IMD 101). For instance, communication unit 208 can be used to send sensor data to controller 300 for additional processing, and/or to receive sensor data from IMD 101. Communication unit 208 comprises one or more RF antennas configured to operate as receivers, transmitters, or transceivers for radio frequency signals. Such wireless links may include Bluetooth™, Bluetooth Low Energy, ultra-wideband, or any other known wireless transmission protocol having suitable authentication and encryption to protect patient data.

In some examples, charger communication unit 208 may be configured to receive an advertising signal and other wireless data from IMD communication unit 108. Such data may include a unique device identifier of IMD 101, a received signal strength indicator (RSSI), an angle of arrival (AoA), or an angle of departure (AoD).

In certain instances, system 10 may use detection of a known device identifier (such as that of the IMD 101, stored in memory) with an RSSI exceeding a predetermined threshold as a trigger to activate the first and second sensors (102, 202). By employing this approach, power is supplied to the sensors (102, 202) only when the rechargeable device (or IMD 101) and a trusted charger 200 are in close proximity, as opposed to when they are a substantial distance apart. This helps ensure that energy is not unnecessarily expended by either the charger 200 or the IMD 101 to power their sensors (102, 202) when sensor-based alignment is not required.

Controller 300 includes a processor 304 that is electrically coupled to a communication unit 308 and a display 307. Processor 304 is configured to process data collected by sensors 102 and 202, or data derived from that collected by sensors 102 and 202, to generate digital twins of the charger 200 and IMD 101 on display 307, providing a user with a visual aid for aligning the devices.

Using inertial sensor data, the respective dispositions of IMD 101 and charger 200 may be determined (i.e. measured or calculated) relative to any of: a global reference frame, a local reference frame, or relative to one another. For example, data collected by inertial sensors 102 and 202 can be used to determine the position and orientation of the IMD 101 and charger 200. A comparison (e.g., via processor 304) of the data can be used to quantify their relative alignment.

Processing the inertial data can involve sensor fusion, whereby inertial data from two or more complementary sensors is algorithmically combined to enhance the accuracy of the system's tracking and alignment capabilities. Any one or a combination of the system's processors (e.g., 104, 204, 304) could be utilized for such purposes. The inertial data may also be computationally transformed (e.g., by any one or a combination of processors 104, 204, 304) into quaternions (e.g. unit quaternions), and algorithms, such as, e.g., a Kalman filter, may also be applied. In exemplary implementations, the controller 300 acts as a central hub for receiving, processing, and fusing sensor data received from IMD 101 and charger 200.

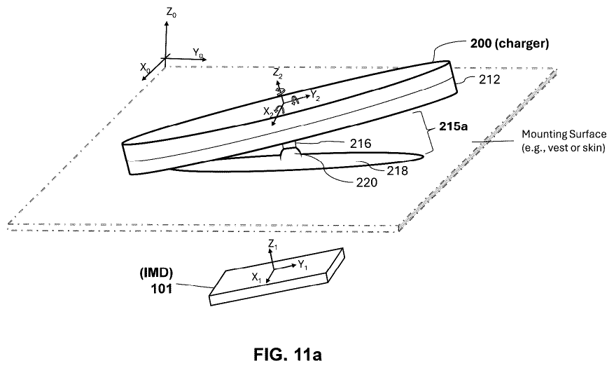

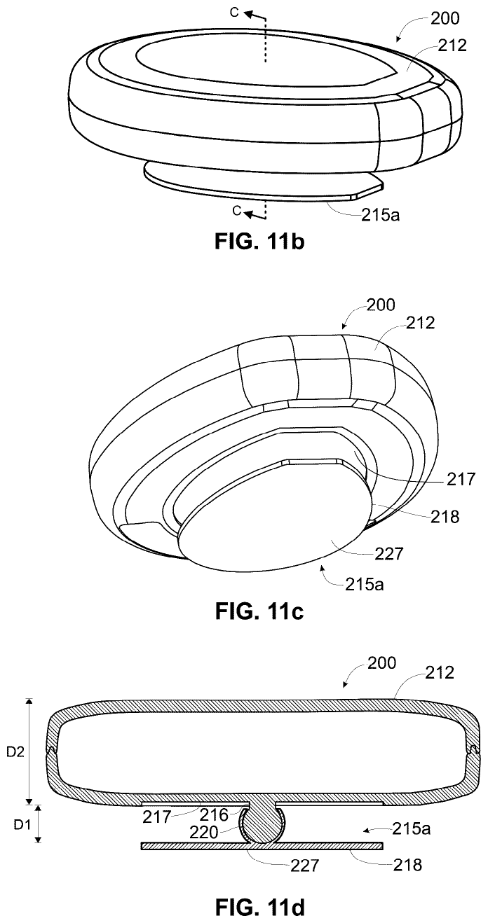

System 10 further includes mount 215 that may be coupled to the charger 200 or formed integrally with housing 212. Mount 215 provides a resiliently movable/tiltable structure that allows the patient to adjust and fix the orientation of charger 200 for optimal alignment with IMD 101. Mount 215 is configured for manual operation to the extent that, during an alignment process, a patient physically manipulates (e.g., moves, tilts, or rotates) mount 215 to make fine-tuned adjustments to the disposition of charger 200 until the transmitting and receiving coils (213 and 113, respectively) are suitably aligned. Three exemplary mounts (215a, 215b, and 215c) are provided in FIGS. 11-13 and will be further discussed in later portions of this disclosure.

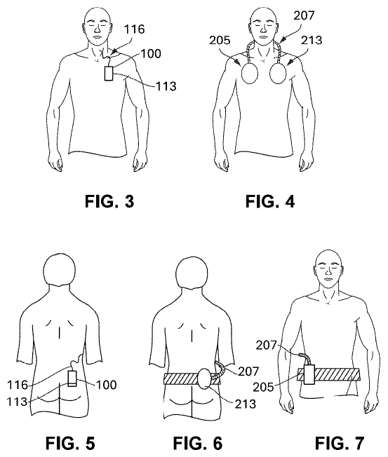

Continuing, FIGS. 3-7 show various examples of systems 10, which include IMD(s) 101 implanted either in the upper torso or lower back of a patient. FIGS. 3-7 are provided to demonstrate a few of many possible ways to initially position charger 200 on a patient before executing a fine-tuned alignment using the digital twins and mount 215. The charger 200 depicted in FIGS. 4, 6, and 7 have a transmitting coil 213 housed separately from an electronics assembly 205, connected by cable 207. Although the electronics assembly 205 does not always need to be housed separately from the transmitting coil 213, doing so can help prevent eddy currents from being induced in the electronic components.

In the configurations depicted in FIGS. 3 and 4, the implantable medical device (IMD) 101 is situated within the patient's torso. The patient is shown wearing a representative charger 200 designed for effective alignment with the IMD 101. In this arrangement, cable 207 connects the transmitting coil 213 with the electronics assembly 205. This cable serves a practical role by allowing the user to position the transmitting coil 213 in proximity to the IMD 101 while maintaining separation from the electronics assembly 205, which may be desirable for usability or safety reasons. For instance, the user can loop cable 207 around the neck so that the transmitting coil 213 rests near the IMD 101 on the left side of the chest. At the same time, the electronics assembly 205 is situated on the right side, ensuring both components are optimally placed for charging without direct overlap.

In the scenarios illustrated in FIGS. 5 through 7, the IMD 101 is implanted in the lower torso, such as the lower back region. For example, the user can wrap cable 207 around either the right or left side of the torso, thereby directing the transmitting coil 213 to a location adjacent to the IMD 101 at the lower back. In these examples, both the transmitting coil 213 and the electronics assembly 205 are designed to be releasably attached to a mounting garment, such as a belt-like structure, which secures the components in place during the charging process.

Other embodiments may not include cable 207, although they still allow the charger 200 to be attached directly to the user's skin, a vest, or other articles of clothing, offering flexibility in placement and ensuring that effective alignment between the charger and the IMD can be achieved across a range of body types and implant locations.

Turning to FIGS. 11a-11d, a first example embodiment for a mount 215a is shown assembled with charger 200. Mount 215a is designed to be manually manipulated (e.g., tilted, rotated, or repositioned) by a patient to align the charger 200 properly with IMD 101. Mount 215a features a shank 216 formed integrally with and extending downwardly from a lower surface 217 of housing 212. An articulating base 218 is positioned beneath shank 216 and is dynamically connected to shank 216 via coupling 220. In this example, coupling 220 comprises a ball joint and allows the charger 200 to be tilted and/or rotated relative to base 218. During charging, the bottom surface 227 of base 218 is placed next to the patient. It can be releasably attached to the patient's skin or clothing, using, for example, hook-and-loop fasteners, non-permanent adhesives, or the like.

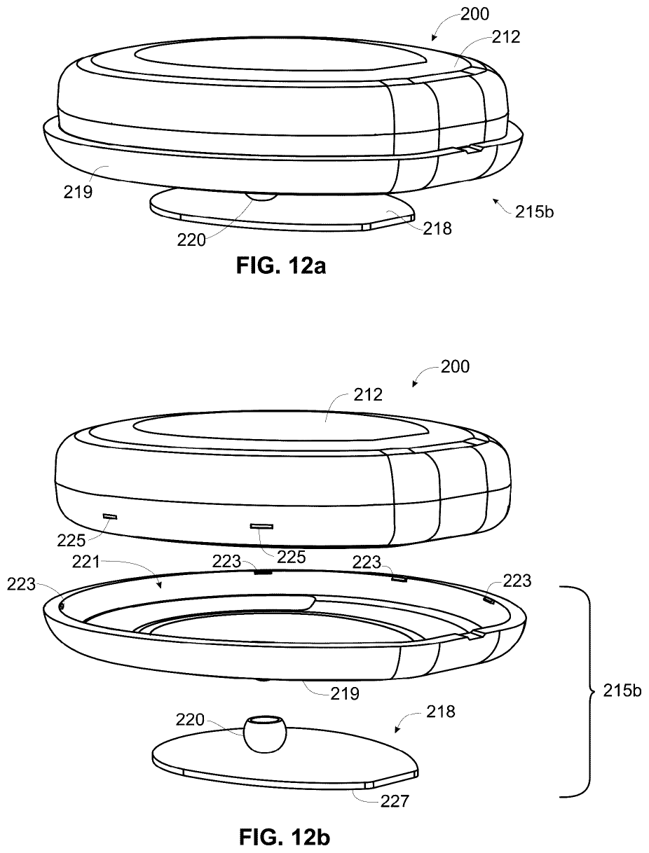

FIGS. 12a-12b provide another example of a mount 215b shown in sub-assembly with charger 200. Mount 215b is similar to mount 215a in that it comprises a ball joint coupling to base 218. However, instead of shank 216 being directly coupled to housing 212, shank 216 (not shown) extends downwardly from an upper platform 219. Platform 219 has a concave upper surface 221 designed to interface with a convex lower surface 222 of housing 212. A plurality of protrusions 223 extend inwardly from a rim of the upper surface 221 to form a snap-fit coupling with a plurality of apertures 225 on the exterior of housing 212. In this manner, mount 215b can be releasably attached to charger 200.

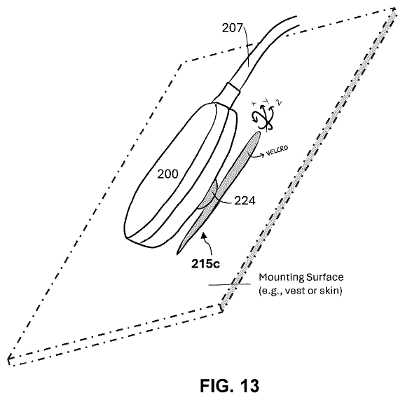

In FIG. 13, a third example of a mount 215c is provided. Mount 215c is not illustrated to include any dynamic couplings or parts that articulate relative to housing 212 of charger 200. Instead, mount 215c features a hemispherical dome 224 that extends outward from the lower surface of housing 212. The outer curved surface of the dome 224 includes means for releasably attaching to the patient's skin or clothing. Pivotal movements of the charger 200 about the dome 224 (of mount 215c) let the user position the charger at various angles. Once the user or patient has identified the preferred pose for aligning the charger 200 with IMD 101, they can secure the charger in place using the releasable attachment means (e.g., hook-and-loop fasteners or adhesives).

The releasable attachment means could comprise any known structure or system suitable for non-permanently coupling an object to the skin or clothing of a patient. For example, hook-and-loop fasteners, adhesives, snaps, suction cups, magnets, or the like could be used. In a circumstance in which the releasable attachment means comprises a form that requires coupling together two or more complementary fasteners (such as, for example, the complementary sides of a hook-and-loop fastener), system 10 may include a mounting garment (like, e.g., the belt shown in FIGS. 6-7) that contains the complementary fastener.

When using inertial sensors for object tracking, it is often necessary to periodically calibrate the inertial sensors to identify and correct inherent sensor errors, such as bias, scale factor errors, and axis misalignments. Without calibration, these initial, deterministic errors and continuous drift over time could lead to inaccuracies in orientation, position, and movement data.

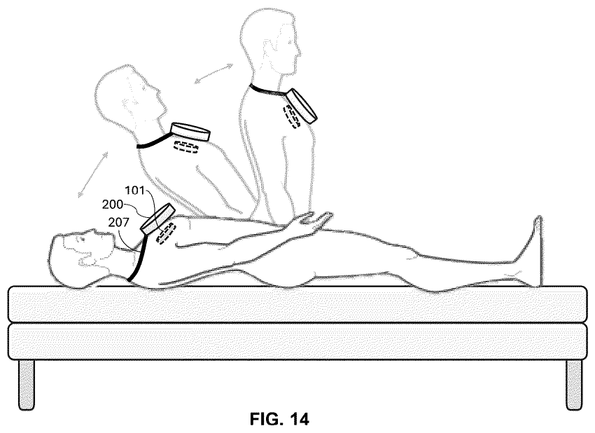

FIGS. 14-17 provide demonstrative images of a patient conducting exemplary methods for calibrating inertial sensors 102 and/or 202. In these examples, system 10 includes an IMD 101 implanted in the patient's torso, and a charger 200 with a cable 207 that drapes around the user's shoulders to initially position the transmitting coil in the vicinity of IMD 101.

In FIG. 14, the IMD 101 and the charger 200 are simultaneously moved through a range of motion, which, in this instance, involves the patient transitioning between a seated position and a reclined position.

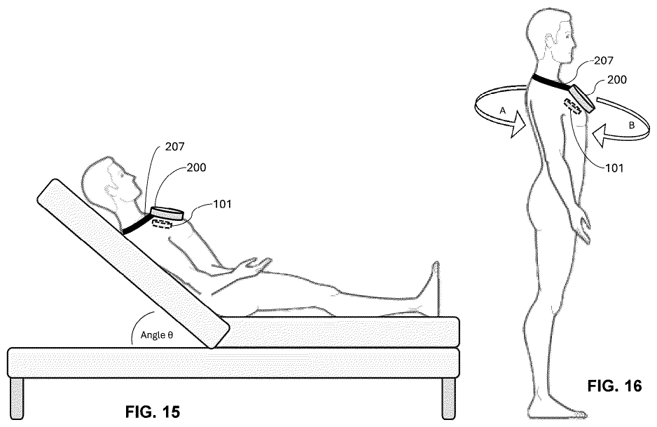

In FIG. 15, an inertial sensor calibration procedure is demonstrated in which the patient is stationarily reclined at a known angle θ, and inertial sensors 102, 202 are stationarily calibrated with reference to the known position or angle of the patient. The angle θ, an angle derived therefrom, or a more generalized indication as to the patient's current body position may be manually entered into a user interface of controller 300. Such data may then be transmitted to any processor within system 10 configured to execute a software calibration procedure, to correct for any offsets or drift in the data.

In FIG. 16, another example (or step) of an inertial calibration procedure may involve instructing the patient to rotate their torso (in the direction of arrows A or B) for the purpose of simultaneously calibrating inertial sensors 102 and 202.

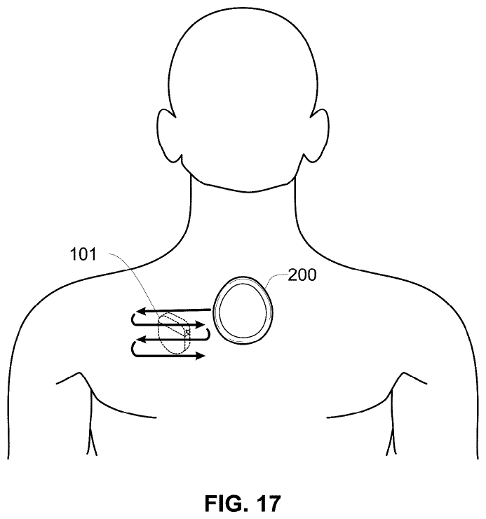

FIG. 17 provides an illustration depicting a patient scanning a charger 200 over the general area of the IMD 101. As this occurs, measurements of current/voltage (e.g., rectified current or voltage in the receiving coil) may be collected continuously and fused with inertial data, enabling triangulation of the location of a relative maximum charge transfer efficiency. Similar methods for tracking charge transfer efficiency (CTE) could be executed by a sensor 202 in the charger 200 configured to monitor the voltage and/or current through the transmitting coil. Such measurements can be indicative of the strength of an inductive coupling and, when fused with IMU data, can allow for precise identification of a location of maximum charging efficiency, and a distance or direction between the charger and said location, which can be presented to the user on a display as an alignment aid.

In alternative examples, inertial sensors in the charger and the IMD could be calibrated at different times, using calibration procedures that differ from those shown and described with reference to FIGS. 14-17.

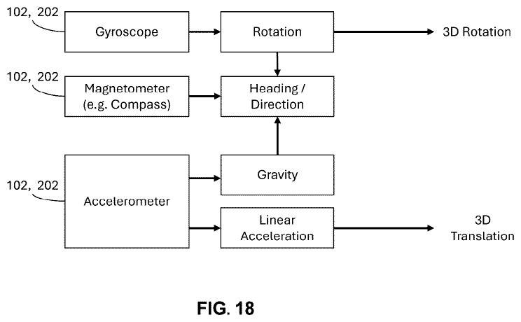

Continuing, FIG. 18 provides a flowchart depicting the general functionality of a sensor fusion processor. A sensor fusion processor accepts inputs from two or more complementary types of sensors and algorithmically combines the complementary data to reduce signal noise and improve the accuracy of pose tracking. As noted above, either or both inertial sensors 102, 202 may comprise two or more complementary types of inertial sensors in certain embodiments, and, in such cases, any one or a combination of processors 104, 204, or 304 may be configured for conducting sensor fusion processing.

For example, in certain embodiments, the charger 200 and the IMD 101 each include an inertial sensor (202 and 102, respectively) that comprises a six-axis IMU including an 3-axis accelerometer and a 3-axis gyroscope. For each device, the gyroscope data (i.e., angular velocity data) is fused with the accelerometer data to calculate the device's orientation. This process is robust for short-term motion tracking, making it sufficient for determining rotational alignment.

The accelerometer outputs can be double-integrated to estimate positional changes of the IMD 101 and charger 200 over time. However, this can, in some implementations, introduce drift due to errors in the accelerometer, making external references (like Bluetooth signals, or measurements of charge transfer efficiency) important for periodic correction. To enhance positional tracking and improve system accuracy in determining when the IMD 101 and charger 200 are axially aligned, the inertial data collected by accelerometers within the IMD 101 may be supplemented by performing charge field mapping, wherein measurements of charge transfer efficiency (CTE), relating to the amount of voltage or current induced in the receiving coil 113, are continuously tracked and fused with inertial data as the user moves charger 200 in the vicinity of IMD 101, similar to as shown in FIG. 17.

The external controller 300 acts as a central hub for processing the accelerometer, gyroscope, and CTE data, and can utilize one or more processing techniques to place the charger 200 and IMD 101 in the same inertial reference frame and to correct for any such errors in the accelerometer positional data, thereby allowing for robust tracking of the relative disposition (i.e. alignment) of the IMD 101 and charger 200.

For instance, a device such as a smartphone could act as the external controller 300, wirelessly receiving inertial data from both the charger 200 and the IMD 101 via Bluetooth or another wireless communication protocol (e.g., Wi-Fi, UWB, NFC, or custom RF communication). The data transfer process would typically involve: (a) data transmission, (b) sensor fusion, and (c) reference frame alignment. During data transmission, the charger 200 and IMD 101 continuously send inertial data to the external controller 300, and the IMD may also send data related to charge field mapping, as discussed above. Each device timestamps its data to ensure synchronization. Next, the external controller applies sensor fusion algorithms (such as an Extended Kalman Filter or complementary filter) to process the data streams. Orientation is computed using gyroscope readings, stabilized with accelerometer and charge-field data to align both devices in a shared reference frame, and relative positions can be inferred by tracking changes in acceleration and integrating motion data over time.

Following calibration and fusion of the inertial data, system 10 is configured to generate graphical representations (i.e., digital twins) of the charger 200 and the IMD 101 on a display 307, providing a user with a visual aid for tracking the dispositions of the charger 200 and the IMD 101 during an alignment process. Depending on the particular needs of a given application, the dispositions of the charger 200 and IMD 101 could also be determined (and subsequently displayed) at times other than the exemplary alignment-for-charging application.

More particularly, the calibrated and fused inertial sensor data is algorithmically processed, e.g., by any of processor 104, 204, 304 (or otherwise) using quaternions, rotation matrices, Euler angles, or mathematical analogs thereto, to transform the inertial data in such a manner as to quantify the dispositions of charger 200 and IMD 101 in 3D space. The use of quaternions may be preferred over other computational methods due to the advantage of avoiding gimbal lock and/or optimizing the memory usage of system 10. However, regardless of the particular algorithm employed, the transformed inertial data is used to generate 3D images (i.e., digital twins) of charger 200 and IMD 101 on display 307. The location and orientation of the digital twins dynamically adjust in real-time as the user moves the charger 200 (and/or a sub-assembly of charger 200 and mount 215), providing visual feedback that allows a user to easily align the charger 200 with the IMD 101 while observing the display 307.

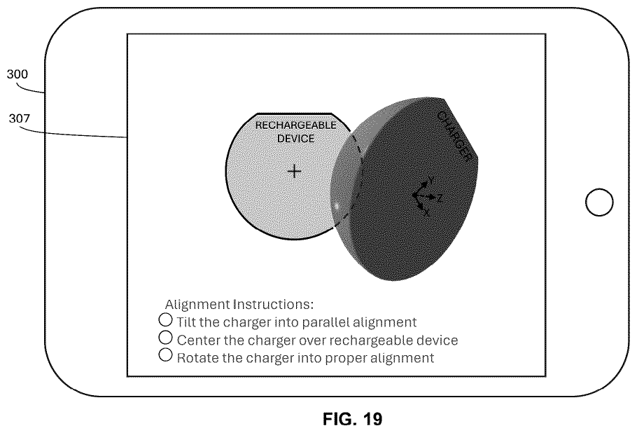

FIGS. 19-22, for example, provide a series of example images showing digital twins generated on a display 307 using the transformed inertial data, and during a process of aligning the charger 200 with IMD 101. These images, generated in real time or near real time, enable users to monitor the relative dispositions of the devices as they move, ensuring precise and efficient alignment. The accuracy of this live tracking depends on the sampling rates of the inertial sensors 102 and/or 202; higher sampling rates allow the system to reflect movements in real time, while lower sampling rates may be employed to conserve memory resources or to capture device positions at specific, discrete moments.

The graphical display 307 serves as a valuable guide for users, enabling informed adjustments and facilitating proper alignment between the charger and IMD. The generation of these images is flexible and can be configured to meet the application's requirements, allowing visual feedback before, during, or after the charging process.

In the given examples, digital representations for the charger 200 and IMD 101 are provided as simplified geometric figures instead of realistic graphical depictions for the sake of convenience and clarity in conveying how the respective devices are intended to align. Those skilled in the art will appreciate that such ornamental aspects are infinitely variable and that the utilitarian aspects of this disclosure, as they relate to FIGS. 19-22 and beyond are therefore not to be misconstrued as limited to any particular ornamental embodiment of the digital twins. As such, a wide variety of shapes, symbols, or depictions could be generated on display 307 using the transformed inertial data, and such images could differ significantly from those of FIGS. 19-22 without extending beyond the intended scope of this disclosure.

In FIGS. 19-22, the digital representation of the charger 200 comprises a 3D hemisphere shape having a single chamfered edge to denote the charger's heading. The rechargeable device is depicted as a 2D image for simplicity, but may, in other cases, comprise a 3D representation.

In other embodiments, all or some of the graphical representations may be provided in 2D or 3D, and, in some instances, multiple orthographic views (e.g. a front view, top view, and a side view) could be provided as an alternative method for guiding a user towards achieving tri-planar alignment between charger 200 and IMD 101.

A series of written alignment instructions are also be provided (see, e.g., lower portions of display 307 in FIGS. 19-22), and these may guide a user's movements of charger 200. In some cases, audible alignment instructions could also (or alternatively) be provided via, e.g., a speaker on the charger 200 or controller 300, and other types of sensory feedback, such as flashing lights, audible beeps, or haptic feedback, could be provided for alerting the user when alignment/misalignment occurs, or for guiding physical movements of charger 200 toward an optimal disposition for charging.

Referring specifically to FIG. 19, a first example image is provided for a series of example images that may be generated on display 307 during a simulated alignment process. The graphical depiction of the charger is shown as being initially misaligned with the rechargeable device, the charger being disposed laterally rightward and angularly askew.

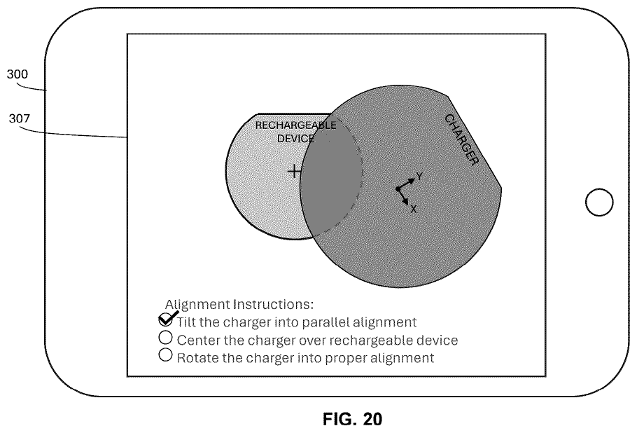

FIGS. 20-22 continue to track the disposition of charger 200 throughout discrete stages of an alignment process as if a user, between each successive image, had manually manipulated the physical charger 200 (and/or mount 215) in accordance with the written alignment instructions provided at the bottom of display 307.

FIG. 20, for example, shows a second example image generated on display 307 after the charger 200 has been successfully tilted into parallel alignment with the rechargeable device (e.g. IMD 101). A status indicator (e.g. a checkmark) is provided beside the written instructions on display 307 to indicate that the charger 200 has been successfully aligned with the IMD 101 along one (of the three, total) orthogonal planes.

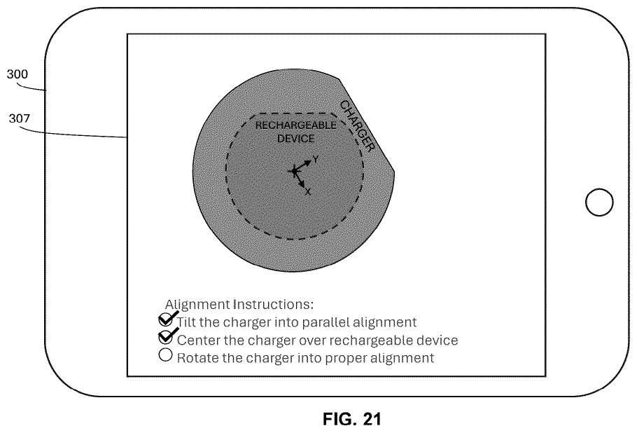

Continuing, FIG. 21 provides a third example image of the series of images generated on display 307 during an alignment process, and, more particularly, the graphical depiction of FIG. 20 is generated sometime after that of FIG. 20 and following translation of the charger 200 (or a subassembly including charger 200 and mount 215) into axial alignment with the rechargeable device. As with FIG. 20, FIG. 21 provides a status indicator (e.g. a checkmark) beside the written instructions to denote that charger 200 has been successfully aligned (with IMD 101) along another plane.

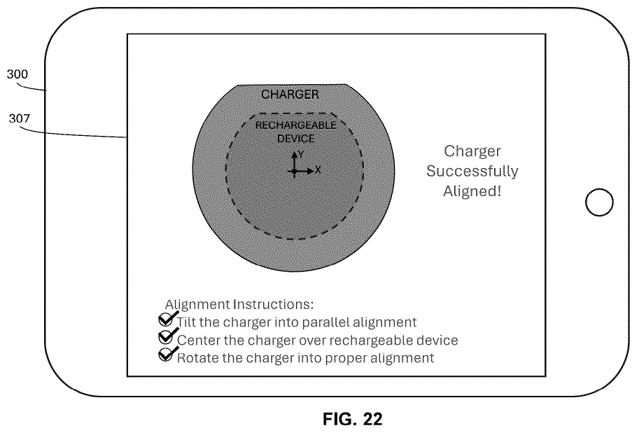

FIG. 22 provides a fourth example image of the series of images generated on display 307 during an alignment process, wherein FIG. 22 is provided as an example of an image that may be generated sometime after that of FIG. 21 and following a user's rotation of charger 200 in accordance with the written alignment instructions to align its heading with that of the rechargeable device. Again, a checkmark is provided to indicate that charger 200 and IMD 101 have been successfully aligned along all three planes.

In various embodiments, any of the images generated in FIGS. 19-22 could be generated in an order different from that which is shown, and additional images may be generated on the display in between any of the successive images shown in FIGS. 19-22.

Further, any one of the processors 104, 204, or 304 could be responsible for generating graphics for display on display 307, and any one or a combination of processors (104, 204, and/or 304) may also be responsible for computationally processing inertial sensor data, for performing inertial data sensor fusion, or for calculating one or more quaternions, rotation matrices, or mathematical analogs thereto for the purpose of generating images on display 307.

In some examples, the system 10 could also include other types of sensors (i.e. in addition to inertial sensors 102 and 202) for the purpose of enhancing or supplementing its ability to track and/or determine the dispositions of charger 200 and IMD 101. For example, other types of sensors that may also be included within system 10 could comprise: one or more optical sensors, such as one or more cameras to observe reference points (e.g., infrared LEDs or retroreflective markers), an ultrasonic sensor, a LIDAR sensor, and/or a GPS sensor, and any one or a combination of these could be included within either of controller 300, charger 200, and/or IMD 101 to suit the needs of a given application.

In other embodiments, different configurations are also possible. For example, certain features and combinations of features described above in connection with specific embodiments can be used in other embodiments and combined with other features as appropriate. Additionally, the present disclosure is not limited to rechargeable devices of the specific types shown. Elements of the wireless transfer system from any of the disclosed examples can be adapted to work with various implantable medical devices, vehicles, consumer electronics, or other types of rechargeable devices.

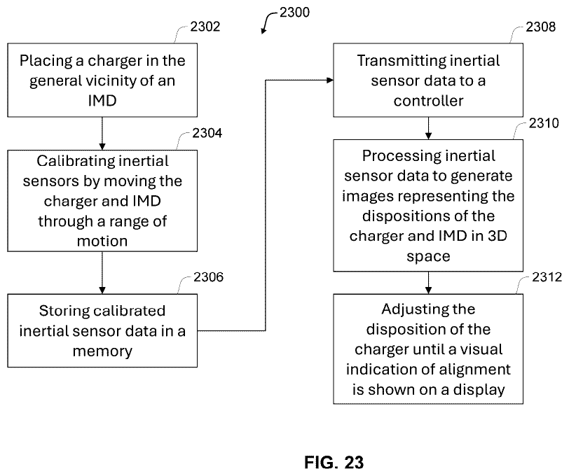

FIGS. 23-25 each provide a flowchart listing a series of steps in accordance with one or more methods. More particularly, FIG. 23 provides a generalized method for aligning a charger with an IMD. FIGS. 23-25 are methods associated with computer-implemented processes executed by one or more processors in system 10.

In FIG. 23, the method comprises: step 2302 for placing a charger 200 in the general vicinity of IMD 101; step 2304 for calibrating inertial sensors (e.g. sensors 102 and/or 202) by moving the charger 200 and/or the IMD 101 through a prescribed range of motion; step 2306 for storing the calibrated inertial sensor data in a memory; step 2308 for processing the inertial sensor data to generate images (i.e., digital twins of the charger and the IMD) on a display, the images representing the position and orientation of the charger 200 and IMD 101 in 3D space; and step 2310 for adjusting, via manual manipulation of the charger 200 and/or mount 215, the disposition of the charger 200 until a visual indication of alignment with IMD 101 is shown on the display 307.

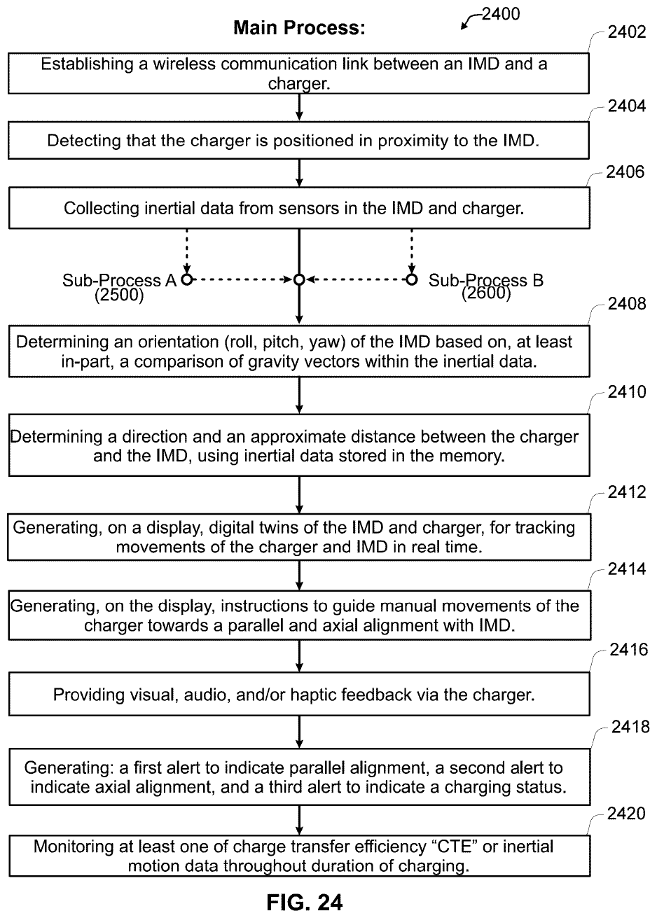

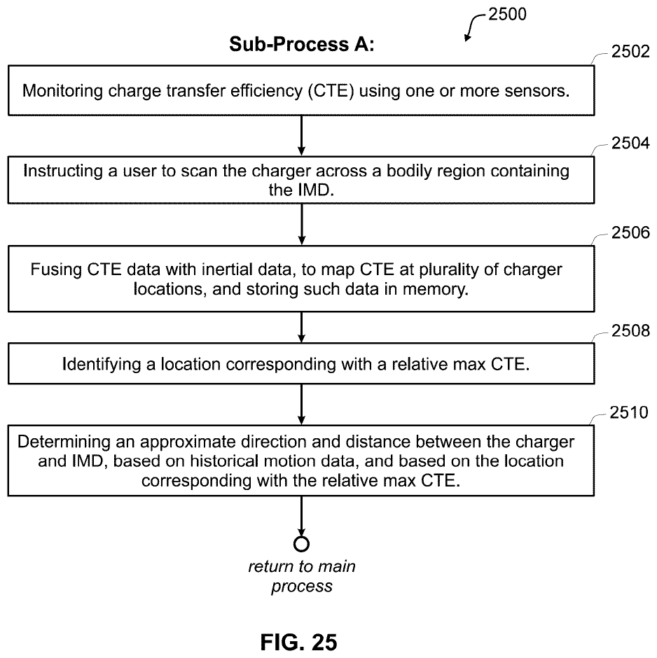

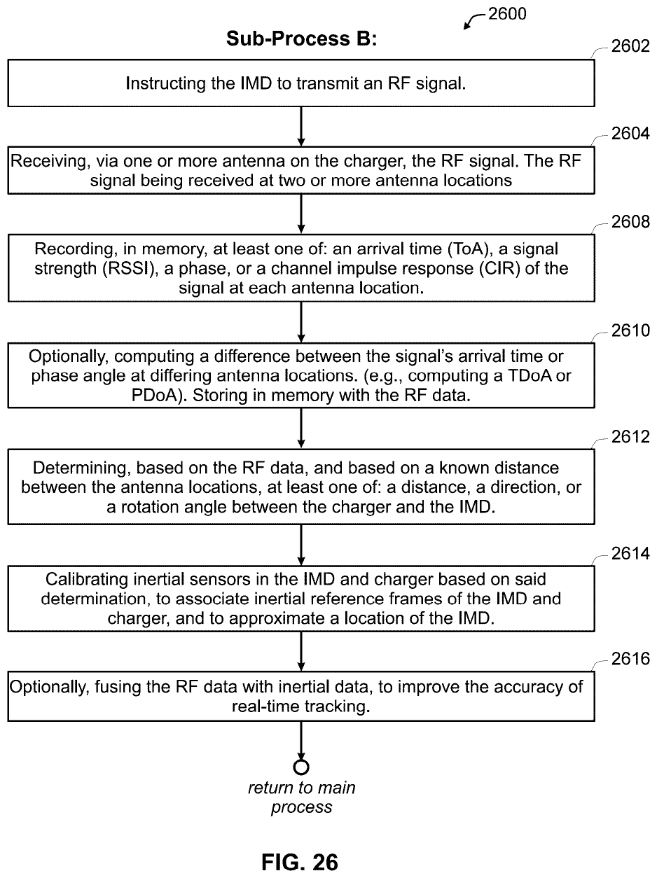

FIGS. 24-26 provide steps for computer-implemented processes (including a main process 2400 and optional sub-processes 2500 and 2600) for collecting and processing data used to generate visual, haptic, and/or audio feedback to aid a user with manual charging alignment. FIG. 24 illustrates a main process 2400 for collecting and processing IMU data and generating digital twins. FIG. 25 relates to sub-process 2500 for collecting and processing charge transfer efficiency (CTE) data, which may be fused with IMU data for triangulating the IMD 101 location. FIG. 26 relates to sub-process 2600 for collecting and processing radio frequency (RF) data, which may be fused with IMU data to help determine a relative direction or distance between charger 200 and IMD 101.

In FIG. 24, step 2402 includes establishing a communication link between IMD 101 and charger 200. In some cases, the IMD 101 may be configured to periodically transmit an advertisement that includes a unique device identifier, which is known to the charger 200 and stored in the charger memory. Upon detection of the unique device identifier and an RSSI that exceeds a predetermined threshold, charger 200 may automatically initiate a handshake to establish a secure communication link with IMD 101. In other examples, the communication link could be established through a different pairing procedure.

Step 2404 comprises detecting that the charger 200 and IMD are positioned in proximity to one another. This could involve, for example, detecting a change in the magnetic field, detecting an RSSI that exceeds the predetermined threshold, or otherwise.

Steps 2406-2408 involve collecting data from inertial sensors in the IMD 101 and charger 200. This is typically performed when the patient is still and each device is static so that gravitational vectors from each device may be collected and compared in step 2408. Typically, a processor in either the charger 200 or the controller 300 would be responsible for computing a transformation matrix to determine an offset between the charger and IMD gravitational vectors. In addition, the output from step 2406 may be transmitted to either or both sub-processes 2500 or 2600, as will be described in later paragraphs. The outputs of these sub-processes (2500 and/or 2600) can be fed back into step 2408 to improve the system's tracking precision.

In step 2410, a system processor (e.g., processor 304) is responsible for processing calibrated and fused inertial data to determine an approximate distance and direction between the charger and the IMD.

Using the sensor data and the approximate distance and direction determined in step 2410, step 2412 involves generating digital twins of the charger and the IMD on a display. Instructions for aligning the charger with the IMD may also be displayed, as noted in step 2414.

Step 2416 provides visual, audio, or haptic feedback to the user as they move the charger in accordance with the alignment instructions in step 2414. These may include beeps, vibrations, or blinking lights provided via a speaker, a mechanical transducer, or a light on the charger, in addition to the visual feedback provided by the digital twins.

In step 2418, various alerts are presented to the patient. These could be provided in the form of lights, vibrations, beeps, or visual alerts on display 307. The various alerts may include a first alert when parallel alignment is achieved, a second alert when axial alignment is achieved, and a third alert to indicate a change in charging status (i.e., when charging is started, stopped, or paused). An alert may also be generated when the charger and IMD are rotationally aligned with the correct heading. All such alerts are optional and could be excluded from some implementations.

In step 2420, after charging has been initiated, one or more inertial signals or charging parameters are monitored throughout the charging process. This is performed so that charging can be paused in response to user movement, misalignment of the coils, or a fully charged IMD battery. For instance, the system may be configured to monitor charge transfer efficiency (CTE) to automatically detect when devices become misaligned, or to monitor inertial data from the IMD or charger to pause charging in response to gross patient movement.

With reference to FIG. 25, sub-process 2500 is provided in accordance with steps 2502-2504 for monitoring charge transfer efficiency (CTE) between charger 200 and IMD 101. CTE data can be optionally fused with inertial sensor data to triangulate the position of the IMD 101 and determine an optimal orientation for the charger 200.

Step 2502 comprises monitoring CTE using one or more sensors in the charger or IMD. For instance, CTE can be monitored on the side of the transmitting coil by using one or more sensors to track the voltage or current through the transmitting coil 213, as either measure can be indicative of the strength of an inductive coupling with receiving coil 113.

Step 2504 comprises instructing a user to slowly scan the charger 200 across a bodily area where the IMD is implanted. This may involve generating written instructions on display 307. As this occurs, step 2506 involves tracking and storing CTE data and inertial data in memory for the purpose of mapping charge transfer efficiency for different positions/orientations of charger 200.

In step 2508, the data collected in step 2506 is analyzed by a processor to identify a location corresponding to the relative maximum CTE.

In step 2510, recent motion data from the charger is analyzed to compute a vector from the charger's current location to the location with the maximum CTE. When this data is returned to the main process 2400 at step 2408, it can be used to display the approximate distance and direction between the digital twins generated on display 307.

With reference to FIG. 26, another optional sub-process 2600 is provided. It relates to analyzing characteristics of an RF signal transmitted from IMD 101 to charger 200 to aid in triangulating the location of IMD 101. For instance, various Bluetooth or UWB receivers are capable of determining an angle or distance to transmitting device based on signal characteristics, such as RSSI or AoA. This data may be periodically used, to recalibrate the inertial sensors as needed, or continuously fused with inertial data to enhance tracking precision.

In step 2602, the IMD is instructed to transmit one or more RF signals from communication unit 108. In step 2604, one or more RF signals are received by at least one antenna on charger communication unit 208.

In instances where the charger includes two or more antennas separated by a known distance, the RF signal may be received at the two or more antennas at slightly different times, based on slight differences in their distance from communication unit 108. In instances where the charger includes only one antenna, a first RF signal is received at the antenna when the charger is at a first location, and a second RF signal is received at the antenna after the charger has been moved to a second location. The distance between the first and second locations may be computed using data from the accelerometer, double-integrated over time.

Step 2608 involves recording, in memory, at least one of: an arrival time (ToA), a signal strength (RSSI), a phase of the signal, or a channel impulse response of the signal at each antenna. The metric(s) recorded and used to triangulate the IMD 101 will vary depending on the computational process employed and can vary between embodiments.

Step 2610 is optional and comprises computing a difference between the RF signal arrival time at two or more spatially separated antennas of the charger communication unit 208. This is generally known as the time difference of arrival or “TDoA”. Alternatively, step 2610 could comprise determining a phase difference of the signal at each antenna, known as the phase difference of arrival or “PDoA”. Either of these measurements can be beneficial for estimating an angle or direction between the charger 200 and the IMD 101.

Step 2612 comprises determining at least one of a distance, a direction, or a rotation angle between the charger 200 and IMD 101 based on the processed RF data.

Step 2614 comprises calibrating inertial sensors in the IMD and charger based on the determined distance, direction, or rotation determined in step 2612. For instance, if an approximate distance and direction are determined in step 2612, the inertial reference frames of the devices can be associated and displayed via the digital twins.

Step 2616 is optional and comprises receiving multiple or continuous RF signals from the IMD, and continuously fusing them with inertial sensor data to smooth real-time tracking.

As noted previously, it will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

What is claimed is:

1. A wireless power transfer system comprising:
a charger including:
a housing;
a transmitting coil contained within the housing and configured, when energized, to inductively couple with a receiving coil in an implantable device;
a first inertial sensor coupled to the housing and configured for collecting first data indicative of a disposition of the charger, and
a first communication unit configured for transmitting the first data to a controller;
a rechargeable device including:
a case;
a second inertial sensor disposed within the case for collecting second data indicative of a disposition of the rechargeable device, and
a second communication unit configured for transmitting the second data to the controller;
the controller comprising:
a processor in communication with a display, wherein following receipt of the first and second data, the processor is configured to generate, on the display, a three-dimensional graphic depicting digital twins of the charger and the rechargeable device.

2. The system of claim 1, wherein the rechargeable device comprises an implantable pulse generator.

3. A system for facilitating alignment between a wireless charger and an implantable medical device, the system comprising:
a charger comprising: a housing, a transmitting coil contained within the housing and configured, when energized, to provide a magnetic charging field, a first sensor for collecting first data, and a first communication unit for wirelessly transmitting the first data;
an implantable medical device (IMD) including a case containing a receiving coil, a second sensor for collecting second data, and a second communication configured for wirelessly transmitting the second data; and
a controller comprising: a third communication unit for receiving the first and second data, and a processor configured for processing the first and second data to determine a disposition of the charger relative to the IMD.

4. The system of claim 3, wherein the processor is further configured to generate, on a display, a three-dimensional representation of the charger.

5. The system of claim 3, wherein the first sensor and the second sensor each comprise at least one of a magnetometer, an accelerometer, a gyroscope, or a combination thereof.

6. The system of claim 3, wherein the controller comprises a consumer electronic device.

7. The system of claim 3, wherein the controller comprises a clinician programmer or a patient remote.

8. The system of claim 3, wherein the IMD comprises an implantable pulse generator having stimulation circuitry contained within the case.

9. The system of claim 3, wherein the transmitting coil comprises a plurality of transmitting coils.

10. The system of claim 3, wherein the processor is configured to computationally fuse data collected from two or more types of inertial sensors.

11. The system of claim 3, wherein the controller includes a memory for storing the first data and the second data.

12. The system of claim 3, wherein, during alignment, the processor is configured to generate, on a display, a three-dimensional representation of the IMD.

13. The system of claim 3, further including a mount coupled to the charger and configured to facilitate positional and angular movements of the charger.

14. The system of claim 13, wherein the mount comprises: a platform coupled to a surface of the housing, a base disposed away from the platform, and a shank movably coupling the platform to the base.

15. The system of claim 14, wherein the base includes means for releasably attaching to a garment.

16. A wireless power transfer system, the system comprising:
a charger including: a power transmitting coil configured when energized to provide a magnetic charging field, a first pair of inertial sensors, and a first communication unit coupled to the first pair of inertial sensors for wirelessly transmitting data from the first pair of inertial sensors to an external controller;
an implantable medical device (IMD) including: a power receiving coil disposed within a case, a second pair of inertial sensors, and a second communication unit coupled to the second pair of inertial sensors for wirelessly transmitting data from the second pair of inertial sensors to the external controller; and
the external controller comprising: a third communication unit for receiving the inertial sensor data, a processor for computationally processing the inertial sensor data into one or more quaternions representing a three-dimensional disposition of the charger, and a display in communication with the processor for displaying digital twins generated using the one or more quaternions.

17. The system of claim 16, wherein the digital twins comprise a visual representation of the three-dimensional disposition of the charger relative to the IMD.

18. The system of claim 16, wherein the first pair of inertial sensors and the second pair of inertial sensors each include an accelerometer and a gyroscope.

19. A method for aligning a charger with an implantable medical device (IMD), wherein the charger and the IMD each include an inertial sensor, the method comprising:
calibrating the inertial sensors by moving the charger and the IMD through a range of motion;
collecting calibrated inertial sensor data and storing the calibrated sensor data in a memory;
continuously processing, via a processor, the calibrated inertial sensor data for comparison with data stored in the memory;
generating, via the processor, digital twins representing a real-time disposition of the charger relative to the IMD;
displaying the digital twins on a display; and
manually adjusting a mount in connection with the charger, until a visual indication of alignment is shown on the display.

20. The method of claim 19, wherein processing the inertial sensor data comprises applying a Kalman filter.

21. The method of claim 19, further comprising releasably attaching the mount to a user.

22. The method of claim 19, further comprising collecting charge transfer efficiency data as the charger is moved through the range of motion, and using the charge transfer efficiency data to determine a location of maximum charging efficiency.

23. The method of claim 19, further comprising processing one or more radio frequency signal metrics to determine a direction between the charger and the IMD.
